(19) United States
(12) Patent Application Publication
(10) Pub. No.: US 2025/0383354 A1
(43) Pub. Date: Dec. 18, 2025

(54) LARGE ONCOSOME SURFACE PROTEINS FOR EARLY IDENTIFICATION OF CLINICALLY SIGNIFICANT PROSTATE CANCER

(71) Applicant: Cedars-Sinai Medical Center, Los Angeles, CA (US)

(72) Inventors: Dolores Di Vizio, Los Angeles, CA (US); Tatyana Vagner, Los Angeles, CA (US)

(73) Assignee: Cedars-Sinai Medical Center, Los Angeles, CA (US)

(21) Appl. No.: 18/878,314

(22) PCT Filed: Jun. 28, 2023
(86) PCT No.: PCT/US2023/069242

§ 371 Date: Dec. 23, 2024

Related U.S. Application Data
(60) Provisional application No. 63/356,406, filed on Jun. 28, 2022.

(51) Int. Cl. G01N 33/574; G01N 33/58

(52) U.S. Cl. CPC G01N 33/57434; G01N 33/57407; G01N 33/57415; G01N 33/582

(57) ABSTRACT

The present invention describes method for the detection of surface proteins on large oncosomes as well as detection of proteins from large oncosomes. Further described are method of isolating large oncosomes based on their surface proteins, prognosticating the cancer based on the proteins, and treatment of cancer.

### CROSS-REFERENCE TO RELATED APPLICATIONS

This application includes a claim of priority under 35 U.S.C. § 119 (e) to U.S. provisional patent application No. 63/356,406, filed Jun. 28, 2022, the entirety of which is hereby incorporated by reference.

### STATEMENT REGARDING FEDERALLY SPONSORED RESEARCH OR DEVELOPMENT

This invention was made with Government support under grant nos. CA234557 and CA218526 awarded by National Institutes of Health. The Government has certain rights in the invention.

### FIELD OF INVENTION

This invention relates to isolating large oncosomes, identification and treatment of prostate cancer.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Prostate cancer (PCa) is the most common non-skin cancer and the second leading cause of cancer death in men in the United States. One of major challenges in PCa is the accuracy predicting resistance to current androgen deprivation therapies (ADT). There is no clinical test to predicting resistance to current androgen-blocking therapies. While prostate biopsy is still the principal diagnostic tool in PCa, they cannot be performed once the organ has been removed, as it happens in patients with a high grade but not metastatic disease, which are the ones that undergo ADT.

It is estimated that one in four men diagnosed with PCa eventually dies from the disease. High grade PCa patients usually respond well to androgen deprivation therapy (ADT); however, despite this treatment, most patients eventually develop resistance to ADT and experience disease progression. Accordingly, there is a need in the art for a clinical tool for the stratification and surveillance of non-metastatic high grade PCa in a non-invasive manner.

### SUMMARY OF THE INVENTION

The following embodiments and aspects thereof are described and illustrated in conjunction with compositions and methods which are meant to be exemplary and illustrative, not limiting in scope.

Various embodiments provide for a method for isolating large oncosomes (LOs), comprising contacting a biological sample obtained from a subject to an agent that specifically binds to one or more surface proteins on the LOs, wherein the one or more surface proteins is selected from the group consisting of: (a) PIGS, CALR, CANX, LMAN2, RPN1, RPN2, VDAC1, STT3A and UGGT1, or (b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or (c) LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2; or (d) any combination of (a), (b), and/or (c); and isolating LOs having the one or more surface proteins.

Various embodiments provide for a method for isolating large oncosomes (LOs), comprising: contacting a biological sample obtained from a subject to an agent that specifically binds to one or more surface proteins on the LOs, wherein the one or more surface proteins is selected from the group consisting of: (a) PIGS, CALR, CANX, LMAN2, RPN1, RPN2, VDAC1, STT3A and UGGT1, or (b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or (c) LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, IRPR1, LEMD2, LMF2, MINPP1, PIGS, PNPLA6, TOR1AIP1, and TOR1AIP2, or (e) TOMM40, SLC25A5, ATP5B, HSPA9, HSPD1, IMMT, and HADHA, or (f) ATP5B, HSPD1, RPN2, SLC25A5, TOMM40, DIP2B, and ERBB2IP; or (g) any combination of (a), (b), (c), (e), and/or (f); and isolating LOs having the one or more surface proteins.

In various embodiments, the method can further comprise identifying the subject as having cancer when one or more surface proteins are detected on the isolated LOs. In various embodiments, the method can further comprise identifying the subject as having prostate cancer, glioma or breast cancer when one or more surface proteins are detected on the isolated LOs.

In various embodiments, the method can further comprise identifying the subject as having advance or aggressive cancer when one or more surface proteins are detected on the isolated LOs. In various embodiments, the method can further comprise identifying the subject as having advance or aggressive prostate cancer, advance or aggressive glioma or advance or aggressive breast cancer when one or more surface proteins are detected on the isolated LOs. In various embodiments, the method can further comprise managing the subject's cancer with an aggressive cancer treatment.

In various embodiments, the one or more surface proteins comprise LAMA5, LAMB2, or both. In various embodiments, the method can further comprise identifying the subject as having stable prostate cancer. In various embodiments, the method can further comprise managing the subject's prostate cancer with watchful waiting.

In various embodiments, the one or more surface proteins comprise RPN2, PIGS, or both. In various embodiments, the method can further comprise identifying the subject as having a high likelihood of having lethal prostate cancer. In various embodiments, the method can further comprise managing the subject's prostate cancer with an aggressive cancer treatment.

In various embodiments, the agent that specifically binds to one or more surface proteins on the LO is an antibody or binding fragment thereof that specifically binds to the one or more surface proteins on the LO, optionally, the antibody or binding fragment thereof is conjugated to a magnetic bead.

In various embodiments, the agent that specifically binds to one or more surface proteins on the LO can be affixed to a substrate.

In various embodiments, the agent that specifically binds to one or more surface proteins on the LO can be conjugated to a label. In various embodiments, the label can be a fluorescent label.

In various embodiments, contacting the biological sample obtained from a subject to an agent that specifically binds to one or more surface proteins on the LO can comprise using a cell sorter. In various embodiments, contacting the biological sample obtained from a subject to an agent that specifically binds to one or more surface proteins on the LO can comprise using flow cytometry.

In various embodiments, the biological sample can be a liquid biopsy sample from the subject. In various embodiments, the biological sample can be a plasma sample from the subject.

Various embodiments provide for a method for detecting the presence of one or more proteins from large oncosomes (LOs), the method comprising: isolating large oncosomes (LOs) from the subject; and detecting the presence one or more proteins from the LOs by contacting an agent that specifically binds to one or more proteins from the LOs, wherein the one or more proteins is selected from the group consisting of: (a) PIGS, CALR, CANX, LMAN2, RPN1, RPN2, VDAC1, STT3A and UGGT1, or (b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or (c) LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2; or (d) any combination of (a), (b), and/or (c).

Various embodiments provide for a method for detecting the presence of one or more proteins from large oncosomes (LOs), the method comprising: isolating large oncosomes (LOs) from the subject; and detecting the presence one or more proteins from the LOs by contacting an agent that specifically binds to one or more proteins from the LOs, wherein the one or more proteins is selected from the group consisting of: (a) PIGS, CALR, CANX, LMAN2, RPN1, RPN2, VDAC1, STT3A and UGGT1, or (b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or (c) LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, IRPR1, LEMD2, LMF2, MINPP1, PIGS, PNPLA6, TOR1AIP1, and TOR1AIP2; or (e) TOMM40, SLC25A5, ATP5B, HSPA9, HSPD1, IMMT, and HADHA, or (f) ATP5B, HSPD1, RPN2, SLC25A5, TOMM40, DIP2B, and ERBB2IP; or (g) any combination of (a), (b), (c), (e), and/or (f).

In various embodiments, the method can further comprise identifying the subject as having cancer when one or more proteins are detected from the isolated LOs. In various embodiments, the method can further comprise identifying the subject as having prostate cancer, glioma or breast cancer when one or more proteins are detected from the isolated LOs.

In various embodiments, the method can further comprise identifying the subject as having advance or aggressive cancer when one or more proteins are detected from the isolated LOs. In various embodiments, the method can further comprise identifying the subject as having advance or aggressive prostate cancer, advance or aggressive glioma or advance or aggressive breast cancer when one or more proteins are detected from the isolated LOs. In various embodiments, the method can further comprise managing the subject's cancer with an aggressive cancer treatment.

In various embodiments, the one or more proteins can comprise LAMA5, LAMB2, or both. In various embodiments, the method can further comprise identifying the subject as having stable prostate cancer. In various embodiments, the method can further comprise managing the subject's prostate cancer with watchful waiting.

In various embodiments, the one or more surface proteins can comprise RPN2, PIGS, or both. In various embodiments, the method can further comprise identifying the subject as a high likelihood of having lethal prostate cancer. In various embodiments, the method can further comprise managing the subject's prostate cancer with an aggressive cancer treatment.

In various embodiments, the agent that specifically binds to one or more proteins from the LO can be an antibody or binding fragment thereof that specifically binds to the one or more proteins in or on the LO, optionally the antibody or binding fragment thereof is conjugated to a magnetic bead.

In various embodiments, the agent that specifically binds to one or more proteins on the LO can be affixed to a substrate.

In various embodiments, the agent that specifically binds to one or more proteins on the LO can be conjugated to a label. In various embodiments, the label can be a fluorescent label.

In various embodiments, contacting the biological sample obtained from a subject to an agent that specifically binds to one or more surface proteins on the LO can comprise using a cell sorter. In various embodiments, contacting the biological sample obtained from a subject to an agent that specifically binds to one or more surface proteins on the LO can comprise using flow cytometry.

In various embodiments, the biological sample can be a liquid biopsy sample from the subject. In various embodiments, the biological sample can be a plasma sample from the subject.

Various embodiments provide for a method of quantifying abundance of a cancer-derived protein signature in extracellular vesicles (EVs), comprising: detecting one or more surface proteins on large oncosomes (LOs), the one or more surface proteins selected from the group consisting of: (a) PIGS, CALR, CANX, LMAN2, RPN1, RPN2, VDAC1, STT3A and UGGT1, or (b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or (c) LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2; or (d) any combination of (a), (b), and/or (c); and quantifying the abundance of the one or more surface proteins.

Various embodiments provide for a method of quantifying abundance of a cancer-derived protein signature in extracellular vesicles (EVs), comprising: detecting one or more surface proteins on large oncosomes (LOs), the one or more surface proteins selected from the group consisting of: (a) PIGS, CALR, CANX, LMAN2, RPN1, RPN2, VDAC1, STT3A and UGGT1, or (b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or (c) LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATPOVOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2; or (e) TOMM40, SLC25A5, ATP5B, HSPA9, HSPD1, IMMT, and HADHA, or (f) ATP5B, HSPD1, RPN2, SLC25A5, TOMM40, DIP2B, and ERBB2IP; or (g) any combination of (a), (b), (c), (e), and/or (f); and quantifying the abundance of the one or more surface proteins.

Various embodiments provide for a method for treating cancer in a subject, comprising: obtaining the results of an analysis one or more proteins from large oncosomes (LO) isolated from a subject, wherein the one or more proteins are selected from the group consisting of (a) PIGS, CALR, CANX, LMAN2, RPN1, RPN2, VDAC1, STT3A and UGGT1, or (b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or (c) LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2; or (d) any combination of (a), (b), and/or (c); and managing the subject's cancer with an aggressive cancer treatment if one or more proteins are detected.

Various embodiments provide a method for treating cancer in a subject, comprising: obtaining the results of an analysis one or more proteins from large oncosomes (LO) isolated from a subject, wherein the one or more proteins are selected from the group consisting of (a) PIGS, CALR, CANX, LMAN2, RPN1, RPN2, VDAC1, STT3A and UGGT1, or (b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or (c) LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2; or (e) TOMM40, SLC25A5, ATP5B, HSPA9, HSPD1, IMMT, and HADHA, or (f) ATP5B, HSPD1, RPN2, SLC25A5, TOMM40, DIP2B, and ERBB2IP; or (g) any combination of (a), (b), (c), (e), and/or (f); and managing the subject's cancer with an aggressive cancer treatment if one or more proteins are detected.

Various embodiments provide for a method for treating prostate cancer in a subject, comprising: detecting one or more surface proteins on large oncosomes (LO) isolated from a subject, wherein the one or more surface proteins are selected from the group consisting of LAMA5, LAMB2, RPN2, PIGS, and combinations thereof; and managing the subject's prostate cancer with watchful waiting if LAMA5, LAMB2, or both are detected, or managing the subject's prostate cancer with an aggressive cancer treatment if RPN2, PIGS, or both are detected.

Various embodiments provide for a method for treating prostate cancer in a subject, comprising: obtaining the results of an analysis one or more surface proteins on large oncosomes (LO) isolated from a subject, wherein the one or more surface proteins are selected from the group consisting of LAMA5, LAMB2, RPN2, PIGS, and combinations thereof; and managing the subject's prostate cancer with watchful waiting if LAMA5, LAMB2, or both are detected, or managing the subject's prostate cancer with an aggressive cancer treatment if RPN2, PIGS, or both are detected.

Various embodiments provide for a method for treating prostate cancer in a subject, comprising: managing the subject's prostate cancer with watchful waiting if LAMA5, LAMB2, or both are detected on large oncosomes (LO) isolated from a subject, or managing the subject's prostate cancer with an aggressive cancer treatment if RPN2, PIGS, or both are detected on large oncosomes (LO) isolated from a subject.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1A shows the proteins enriched in LO, HSPA5 and KT18, are present in the 2.8K and 10K, while the proteins enriched in exosomes/small EVs, TSG101, CD81, and CD9 are present in the 100K. CAV1, which is expressed in EVs of different sizes and origin, was detected across all EV populations, and the intracellular marker CYC1 was detected only in the producing cells (WCL) an absent in the EVs.

FIG. 1B shows particle concentration quantified by Tunable Resistive Pulse Sensing (TRPS, qNano) showed that, in each cell line, large EVs in the 2.8K and 10K pellets were 3-4 orders of magnitude less abundant than small EVs in the 100K pellets. The TRPS measurement of EV size distributions in each EV population showed very similar mode and mean diameters across the three cells lines. The inserts show transmission electron microscopy images confirming the enrichment of large EVs in the 2.8K and 10K, and small EVs in the 100K.

FIG. 1C shows the characterization of the EV protein cargo using an untargeted label-free mass spectrometry analysis of the three EV populations from PC3, U87, and MDA-MB-231 cells. The proteomic analysis revealed that, in each cell line, about one half of the detected proteins were common to all three EV populations.

FIG. 1D shows proteins detected in all three cell lines were used to compare their protein contents. Pearson's correlation was used to calculate the correlation matrix and divisive hierarchical clustering (DIANA) was used for ordering samples and proteins.

FIG. 1E show the gene set enrichment analysis (GSEA) of the proteins identified in 2.8K, 10K, and 100K using the LO signature identified in our previous study demonstrated an enrichment of the LO proteins in the 2.8K and 10K EVs in comparison to the 100K EVs.

FIG. 1F shows the selection of proteins unique to or differentially enriched in the 2.8K compared to the 100K EVs.

FIG. 1G shows the proteins that are present on the surface of EVs.

FIG. 1H shows additional proteins that are present on the surface of prostate cancer derived LOs

FIG. 2 shows proteins as markers of large oncosomes that are present on the surface of the vesicles. They are more abundant in LO than small EVs in three different cell lines (PC3, U87 and MDA-MB-231), used as models of prostate cancer, glioma and breast cancer, respectively.

FIG. 3 shows proteins (TOMM40, SLC25A5, ATP5B, HSPA9, HSPD1, IMMT, HADHA) identified as enriched in large oncosomes versus Small-EVs. Additionally these are enriched in large oncosomes also at the RNA level, and PCTA analysis demonstrated that these mRNAs are significantly upregulated in primary tumors in comparison to the benign tissues and further upregulated in metastatic castration resistant prostate cancer (mCRPC) in comparison with non metastatic disease.

FIG. 4 shows that plasma LO from mCRPC patients are a rich source of protein/RNA biomarkers. Left: SWATH-MS identifies LO proteins differentially expressed among short and long prostate cancer patient responders to therapy. Center: LO proteins enriched also at the mRNA level in PC cell line-and patient plasma-derived LO. Right: LO surface proteins identified by MS in both PC cell line-and patient plasma-derived LO.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} e d., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

“Lethal” as used herein in reference to prostate cancer indicates a patient who will die from prostate cancer, not just with prostate cancer.

The term “extracellular vesicles” or “EV” as used herein include any vesicles that are shed by the cells of any living organisms (for example, humans). EVs include but are not limited to exosomes, which are about 20 nm to about 80 nm in size, large oncosomes which are about lum to about 30 μm in size (with a predominance of vesicles of around 3-4 μm) and microvesicles which are about 0.5 to 1 mm in size.

Examples of biological samples include but are not limited to liquid biopsies, body fluids, whole blood, plasma, serum, intestinal fluids or aspirate, and stomach fluids or aspirate, cerebral spinal fluid (CSF), urine, sweat, saliva, tears, pulmonary secretions, breast aspirate, prostate fluid, seminal fluid, intraocular fluid, mucous, and moisture in breath.

The invention described herein provides a strategy for a non-invasive biopsy of circulating EVs from prostate cancer patients. This provides a clinical tool for the stratification and surveillance of non-metastatic high grade PCa in a non-invasive manner.

Quantitative proteomic analysis of the cancer-derived EVs obtained from prostate cancer, glioma, and breast cancer cell lines, as well as from prostate cancer patient plasma, identified a number of proteins in different EV populations. Using our proteomic data and a published database of surface proteins, we identified a list of proteins that are present on the surface of large oncosomes and other cancer-derived EVs. These proteins can be used for affinity immunocapture and isolation of cancer-derived EVs from biofluids, and the molecular cargo of these EVs can be further interrogated. In addition, these surface proteins themselves have prognostic or/and diagnostic value for stratifying the patients with prostate cancer.

We found a set of proteins specific to different EV populations from prostate cancer cells and other tumor cells. The signature will enable capture of cancer-specific EVs from biofluids.

MS-based proteomics profiling continues one of the most widely used approaches to unbiased discovery of novel biomarkers. Nevertheless, a major challenge in identifying highly diluted cancer biomarkers in plasma is the presence of very abundant blood proteins, such as albumins and globulins which dramatically reduce sensitivity. Our approach described herein to isolate circulating L-EVs (i.e., LO) from, for example, plasma of PCa patients can improve current tests for PCa prognosis.

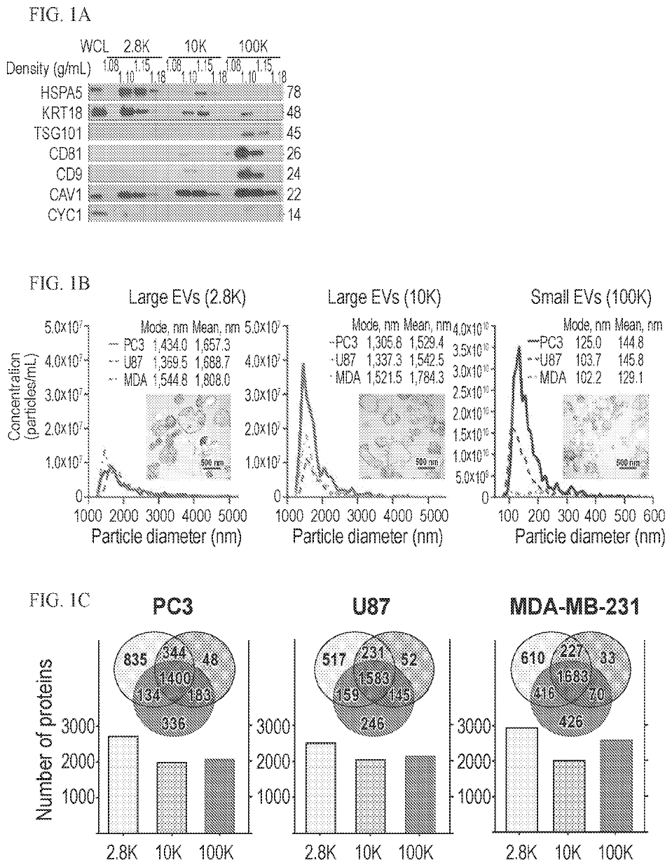

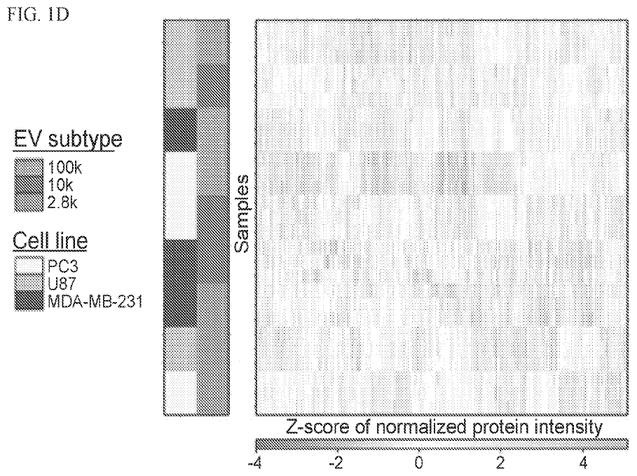

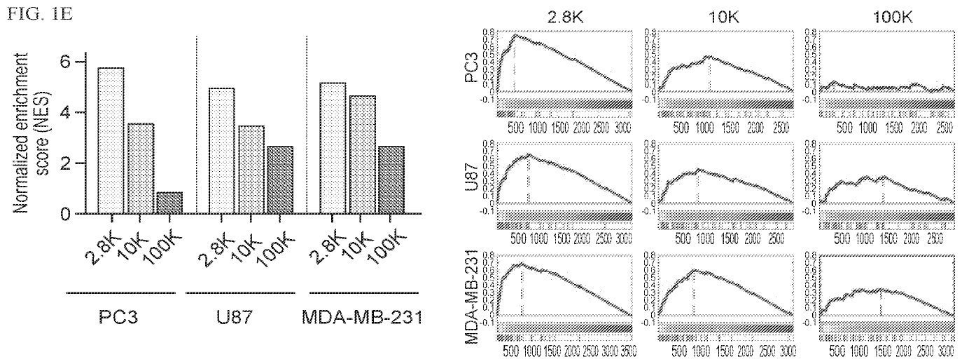

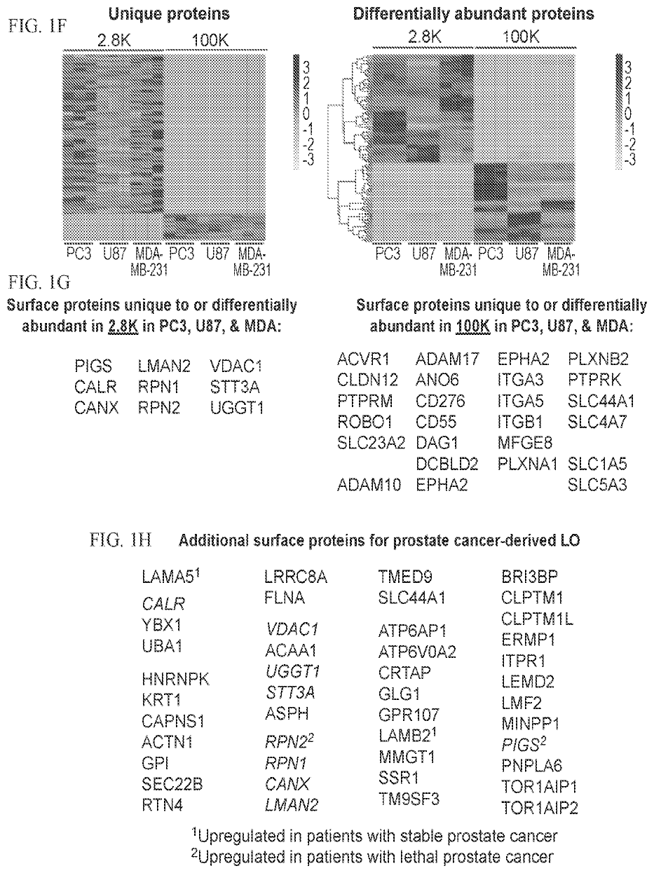

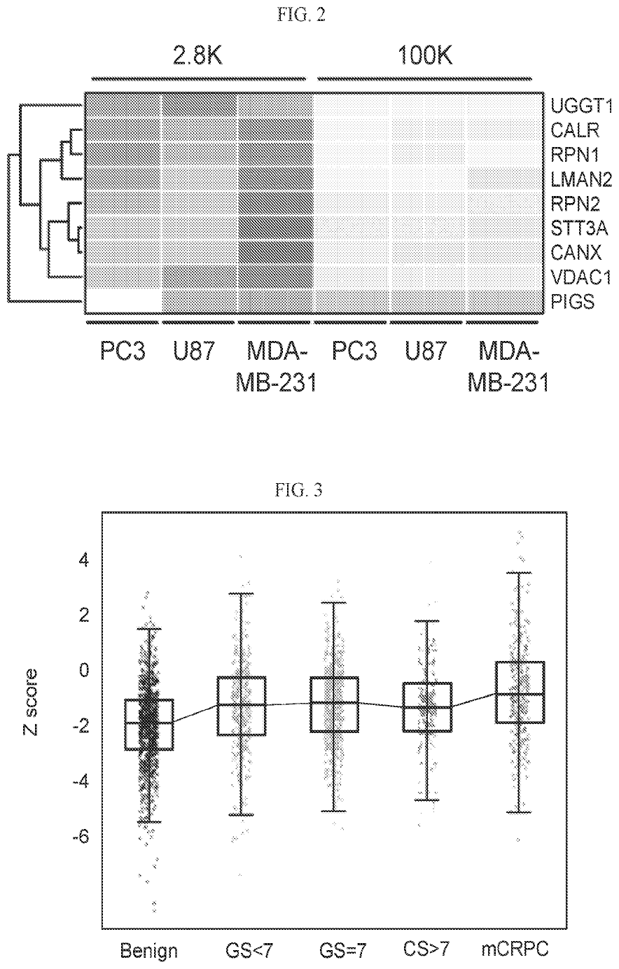

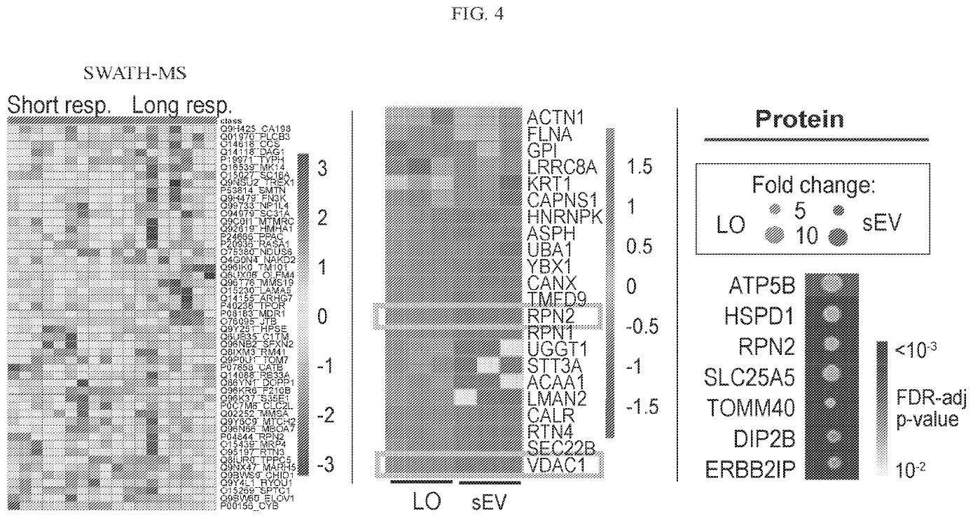

We have previously reported that a population of cancer-specific extracellular vesicles (EVs), large oncosomes (LO), can be isolated by differential centrifugation from conditioned cell culture media and human plasma at 10,000×g. We noticed that large EVs in the size range of LO can also be pelleted at a lower centrifugation speed of 2,800×g, and that this pellet is enriched in particularly large EVs, 4-10 μm diameter, in comparison with the pellet obtained at 10,000×g. We optimized our EV isolation protocol to collect two large EV populations—pelleted at 2,800×g (2.8K) and at 10,000×g (10K),—and a small EV population pelleted at 100,000×g (100K) and demonstrated that the proteins enriched in LO, HSPA5 and KT18, were present in the 2.8K and 10K pellets, and the proteins typically enriched in exosomes/small EVs, TSG101, CD81, and CD9 were present in the 100K pellet. (FIG. 1A).

We isolated, purified by iodixanol density gradient, and characterized the 2.8K, 10K, and 100K EV populations from three different cancer cell lines: prostate cancer (PC3), glioma (U87), and breast cancer (MDA-MB-231). Particle concentrations and size distributions in the EV preparations were quantified by Tunable Resistive Pulse Sensing (TRPS, qNano) and were found to be similar across the three cells lines (FIG. 1B). Transmission electron microscopy confirmed that the 2.8K and 10K EV preparations were enriched in large EVs and the 100K preparation was enriched in small EVs (FIG. 1B, inserts).

To characterize the EV protein cargo, we performed an untargeted label-free mass spectrometry analysis of the three EV populations from PC3, U87, and MDA-MB-231 cells. The proteomic analysis revealed that, in each cell line, about one half of the detected proteins were common to all three EV populations (FIG. 1C). However, each EV population was also characterized by a set of proteins unique to it and a set of proteins shared with one other EV population. The 2.8K EVs had the highest number of proteins identified in it across all three cell lines. The 2.8K EVs from different cell lines also clustered together, based on their EV type, and not based on their cell line of origin (FIG. 1D), suggesting the similarity of their protein composition across the cancer types. In our previous study, we performed comparative proteomic profiling of prostate cancer-derived LO and exosomes and identified a number of proteins deferentially enriched in LO. Using this LO signature, we performed the gene set enrichment analysis (GSEA) of the proteins identified in 2.8K, 10K, and 100K. The GSEA confirmed the enrichment of the LO proteins in the 10K EVs in comparison to the 100K EVs and demonstrated that the LO signature was even more prominent in the 2.8K (FIG. 1E), confirming that LO can be isolated at 2,800×g, and suggesting that they may be more enriched in the 2.8K EV population than in the 10K.

Next, we used our proteomic dataset to identify the cancer EV-specific proteins shared by all three cancer types. We first selected the proteins unique to or differentially enriched in the 2.8K compared to the 100K EVs (FIG. 1F) and among those, we identified the proteins that are present on the surface of EVs (FIG. 1G) using a surface protein database. Since these proteins are exposed on the EV surface, we described herein their use to specifically isolate cancer-derived EVs by, for example, affinity immunocapture from human plasma or/and other biofluids for cancer prognostics/diagnostics.

Over the years, our group has been working on the development of EV liquid biopsy approaches for prostate cancer aimed at early identification of clinically significant cancer and disease progression, therefore, we also performed an untargeted label-free mass spectrometry analysis of LO isolated from plasma of prostate cancer patients with either stable (n=9) or lethal (n=11) disease. Using the obtained dataset, we identified an extended list of surface protein candidates specific to prostate cancer-derived LO (H). These proteins can also be used, for example, for affinity immunocapture and isolation of prostate cancer-derived EVs from biofluids, and the molecular cargo of these EVs can be further interrogated. In addition, some of these surface proteins themselves (such as LAMA5, LAMB2, RPN2, and PIGS) can have prognostic or/and diagnostic value for stratifying the patients with prostate cancer.

As such, various embodiments of the present invention are based, at least in part, on these findings.

### Capture/Isolation

Various embodiments of the invention provide for a method for isolating large oncosomes (LOs), comprising contacting a biological sample obtained from a subject to an agent that specifically binds to one or more surface proteins on the LOs, wherein the one or more surface proteins is selected from the group consisting of: (a) PIGS, CALR, CANX, LMAN2, RPN1, RPN2, VDAC1, STT3A and UGGT1, or (b) ACVR1,CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or (c) LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2; or (d) any combination of (a), (b), and/or (c); and isolating LOs having the one or more surface proteins.

Various embodiments of the invention provide for a method for isolating large oncosomes (LOs), comprising contacting a biological sample obtained from a subject to an agent that specifically binds to one or more surface proteins on the LOs, wherein the one or more surface proteins is selected from the group consisting of: (a) PIGS, CALR, CANX, LMAN2, RPN1, RPN2, VDAC1, STT3A and UGGT1, or (b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or (c) LAMA5, CALR, YBX1, UBAI, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2, or (e) TOMM40, SLC25A5, ATP5B, HSPA9, HSPD1, IMMT, and HADHA, or (f) ATP5B, HSPD1, RPN2, SLC25A5, TOMM40, DIP2B, and ERBB2IP; or (g) any combination of (a), (b), (c), (e), and/or (f); and isolating LOs having the one or more surface proteins.

In various embodiments, the biological sample was pelleted at about 2,800×g (2.8K) or at about 10,000×g (10K). In various embodiments, the biological sample was pelleted at about 2,800×g (2.8K) to 10,000×g (10K). In various embodiments, the biological sample was pelleted at about 2,500×g, about 5,000×g, about 7500×g, about 10,000×g, or about 12,500×g.

In various embodiments, the one or more surface proteins is selected from the group consisting of: any combination of (a), (b), and/or (c). In various embodiments, the one or more surface proteins is selected from the group consisting of: any combination of (e), and/or (f).

In various embodiments, the one or more surface proteins is selected from the group consisting of: PIGS, CALR, CANX, LMAN2, RPN1, RPN2, VDAC1, STT3A and UGGT1. In various embodiments, the one or more surface proteins is 2, 3, 4, 5, 6, 7, 8, or 9 of these proteins.

In various embodiments, the one or more surface proteins is selected from the group consisting of: ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3. In various embodiments, the one or more surface proteins is 2, 3, 4, 5, 6, 7, 8, 9, 10, 11, 12, 13, 14, 15, 16, 17, 18, 19, 20, 21, 22, 23, or 24 of these proteins.

In various embodiments, the one or more surface proteins is selected from the group consisting of: LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2. In various embodiments, the one or more surface proteins is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36,37, 38, 39, 40, 41, 42, 43, 44 or 45 of these proteins.

In various embodiments, the one or more surface proteins is selected from the group consisting of: TOMM40, SLC25A5, ATP5B, HSPA9, HSPD1, IMMT, and HADHA. In various embodiments, the one or more surface proteins is 2, 3, 4, 5, 6, or 7 of these proteins.

In various embodiments, the one or more surface proteins is selected from the group consisting of: ATP5B, HSPD1, RPN2, SLC25A5, TOMM40, DIP2B, and ERBB2IP. In various embodiments, the one or more surface proteins is 2, 3, 4, 5, 6, or 7 of these proteins.

In various embodiments, the method further comprises identifying the subject as having cancer when one or more surface proteins are detected on the isolated LOs. In various embodiments, the method further comprises identifying the subject as having prostate cancer, glioma or breast cancer when one or more surface proteins are detected on the isolated LOs.

In various embodiments, the method further comprises identifying the subject as having advance or aggressive cancer when one or more surface proteins are detected on the isolated LOs. In various embodiments, the method further comprises identifying the subject as having advance or aggressive prostate cancer, advance or aggressive glioma or advance or aggressive breast cancer when one or more surface proteins are detected on the isolated LOs. For example, if the subject has prostate cancer, the detection of the one or more surface proteins identifies the subject as having advance or aggressive prostate cancer; if the subject has glioma, the detection of the one or more surface proteins identifies the subject as having advance or aggressive glioma, if the subject has breast cancer, the detection of the one or more surface proteins identifies the subject as having advance or aggressive breast cancer. In various embodiments, the method further comprises managing the subject's cancer with an aggressive cancer treatment. In various embodiments, managing the subject's cancer with an aggressive cancer treatment comprises administering the aggressive cancer treatment. In certain embodiments, wherein the subject's cancer is managed with an aggressive cancer treatment, the one or more surface proteins detected do not solely comprise LAMA5, LAMB2, or both.

In various embodiments, the one or more surface proteins comprise LAMA5, LAMB2, or both. In various embodiments, the method further comprises identifying the subject as having stable prostate cancer. In various embodiments, the method further comprises managing the subject's prostate cancer with watchful waiting.

In various embodiments, the one or more surface proteins comprise RPN2, PIGS, or both. In various embodiments, the method further comprises identifying the subject as a high likelihood of having lethal prostate cancer. In various embodiments, the method further comprises managing the subject's prostate cancer with an aggressive cancer treatment. In various embodiments, managing the subject's cancer with an aggressive cancer treatment comprises administering the aggressive cancer treatment. Examples of aggressive cancer treatment include but are not limited to first and second line androgen deprivation therapy (ADT) and chemotherapy. An example of chemotherapy includes but is not limited to docetaxel.

In various embodiments, the agent that specifically binds to the one or more surface proteins on the LO is an antibody or binding fragment thereof that specifically binds to the one or more surface proteins on the LO.

In various embodiments, the agent that specifically binds to the one or more surface proteins on the LO is a magnetic particle (e.g., magnetic bead) conjugated to an antibody or binding fragment thereof that specifically binds to the one or more surface proteins on the LO.

In various embodiments, the agent that specifically binds to the one or more surface proteins on the LO is an antibody or binding fragment thereof that specifically binds to the one or more surface proteins on the LO, and the method further uses a microfluidic device to isolate the LO.

In various embodiments, the agent that specifically binds to one or more surface proteins on the LO is affixed to a substrate.

In various embodiments, the agent that specifically binds to one or more surface proteins on the LO is conjugated to a label. In various embodiments, the label is a fluorescent label.

In various embodiments, contacting the biological sample obtained from a subject to an agent that specifically binds to one or more surface proteins on the LO comprises using a cell sorter. In various embodiments, isolating the one or more surface proteins on the LO comprises using a cell sorter.

In various embodiments, contacting the biological sample obtained from a subject to an agent that specifically binds to one or more surface proteins on the LO comprises using flow cytometry. In various embodiments, isolating the one or more surface proteins on the LO comprises using flow cytometry.

In various embodiments, the biological sample is a liquid biopsy sample from the subject. In various embodiments, the biological sample is a plasma sample from the subject. In various embodiments, the biological sample is a serum sample from the subject.

### Detection

Various embodiments of the invention provide for a method for detecting the presence of one or more proteins from large oncosomes (LOs), the method comprising: isolating large oncosomes (LOs) from the subject; and detecting the presence one or more proteins from the LOs by contacting an agent that specifically binds to one or more proteins from the LOs, wherein the one or more proteins is selected from the group consisting of: (a) PIGS, CALR, CANX, LMAN2, RPN1, RPN2, VDAC1, STT3A and UGGT1, or (b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or (c) LAMA5, CALR, YBX1, UBAI, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2; or (d) any combination of (a), (b), and/or (c).

Various embodiments of the invention provide for a method for detecting the presence of one or more proteins from large oncosomes (LOs), the method comprising: isolating large oncosomes (LOs) from the subject; and detecting the presence one or more proteins from the LOs by contacting an agent that specifically binds to one or more proteins from the LOs, wherein the one or more proteins is selected from the group consisting of: (a) PIGS, CALR, CANX, LMAN2, RPN1, RPN2, VDAC1, STT3A and UGGT1, or (b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or (c) LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2, or (e) TOMM40, SLC25A5, ATP5B, HSPA9, HSPD1, IMMT, and HADHA, or (f) ATP5B, HSPD1, RPN2, SLC25A5, TOMM40, DIP2B, and ERBB2IP; or (g) any combination of (a), (b), (c), (e), and/or (f).

In various embodiments, isolating the LO from the subject comprises pelleting a biological sample obtained from the subject at about 2,800×g (2.8K) or at about 10,000×g (10K). In various embodiments, isolating the LO from the subject comprises pelleting a biological sample obtained from the subject at about 2,800×g (2.8K) to 10,000×g (10K). In various embodiments, isolating the LO from the subject comprises pelleting a biological sample obtained from the subject at about 2,500×g, about 5,000×g, about 7500×g, about 10,000×g, or about 12,500×g.

In various embodiments, the one or more proteins is selected from the group consisting of: any combination of (a), (b), and/or (c).

In various embodiments, the one or more surface proteins is selected from the group consisting of: any combination of (e), and/or (f).

In various embodiments, the one or more proteins is selected from the group consisting of: PIGS, CALR, CANX, LMAN2, RPN1, RPN2, VDAC1, STT3A and UGGT1. In various embodiments, the one or more proteins is 2, 3, 4, 5, 6, 7, 8, or 9 of these proteins.

In various embodiments, the one or more proteins is selected from the group consisting of: ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3. In various embodiments, the one or more proteins is 2, 3, 4, 5, 6, 7, 8, 9, 10, 11, 12, 13, 14, 15, 16, 17, 18, 19, 20, 21, 22, 23, or 24 of these proteins.

In various embodiments, the one or more proteins is selected from the group consisting of: LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2. In various embodiments, the one or more proteins is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36,37, 38, 39, 40, 41, 42, 43, 44 or 45 of these proteins.

In various embodiments, the one or more surface proteins is selected from the group consisting of: TOMM40, SLC25A5, ATP5B, HSPA9, HSPD1, IMMT, and HADHA. In various embodiments, the one or more surface proteins is 2, 3, 4, 5, 6, or 7 of these proteins.

In various embodiments, the one or more surface proteins is selected from the group consisting of: ATP5B, HSPD1, RPN2, SLC25A5, TOMM40, DIP2B, and ERBB2IP. In various embodiments, the one or more surface proteins is 2, 3, 4, 5, 6, or 7 of these proteins.

In various embodiments, the method further comprises identifying the subject as having cancer when one or more surface proteins are detected on the isolated LOs. In various embodiments, the method further comprises identifying the subject as having prostate cancer, glioma or breast cancer when one or more surface proteins are detected on the isolated LOs.

In various embodiments, the method further comprises identifying the subject as having advance or aggressive cancer when one or more proteins are detected from the isolated LOs. In various embodiments, the method further comprises identifying the subject as having advance or aggressive prostate cancer, advance or aggressive glioma or advance or aggressive breast cancer when one or more proteins are detected from the isolated LOs. For example, if the subject has prostate cancer, the detection of the one or more surface proteins identifies the subject as having advance or aggressive prostate cancer; if the subject has glioma, the detection of the one or more surface proteins identifies the subject as having advance or aggressive glioma, if the subject has breast cancer, the detection of the one or more surface proteins identifies the subject as having advance or aggressive breast cancer.

In various embodiments, the method further comprises managing the subject's cancer with an aggressive cancer treatment. In various embodiments, managing the subject's cancer with an aggressive cancer treatment comprises administering the aggressive cancer treatment. In certain embodiments, wherein the subject's cancer is managed with an aggressive cancer treatment, the one or more proteins detected do not solely comprise LAMA5, LAMB2, or both.

In various embodiments, the one or more proteins comprise LAMA5, LAMB2, or both. In various embodiments, the method further comprises identifying the subject as having stable prostate cancer. In various embodiments, the method further comprises managing the subject's prostate cancer with watchful waiting.

In various embodiments, the one or more surface proteins comprise RPN2, PIGS, or both. In various embodiments, the method further comprises identifying the subject as having a high likelihood of having prostate cancer. In various embodiments, the method further comprises managing the subject's prostate cancer with an aggressive cancer treatment. In various embodiments, managing the subject's cancer with an aggressive cancer treatment comprises administering the aggressive cancer treatment. Examples of aggressive cancer treatment include but are not limited to first and second line androgen deprivation therapy (ADT) and chemotherapy. An example of chemotherapy includes but is not limited to docetaxel.

In various embodiments, the agent that specifically binds to one or more proteins from the LO is an antibody or binding fragment thereof that specifically binds to the one or more proteins in or on the LO.

In various embodiments, the agent that specifically binds to the one or more surface proteins on the LO is a magnetic particle (e.g., magnetic bead) conjugated to an antibody or binding fragment thereof that specifically binds to the one or more surface proteins on the LO.

In various embodiments, the agent that specifically binds to the one or more surface proteins on the LO is an antibody or binding fragment thereof that specifically binds to the one or more surface proteins on the LO, and the method further uses a microfluidic device to isolate the LO.

In various embodiments, the agent that specifically binds to one or more proteins on the LO is affixed to a substrate.

In various embodiments, the agent that specifically binds to one or more proteins

on the LO is conjugated to a label.

In various embodiments, the label is a fluorescent label.

In various embodiments, contacting the biological sample obtained from a subject to an agent that specifically binds to one or more surface proteins on the LO comprises using a cell sorter. In various embodiments, isolating the one or more surface proteins on the LO comprises using a cell sorter.

In various embodiments, contacting the biological sample obtained from a subject to an agent that specifically binds to one or more surface proteins on the LO comprises using flow cytometry. In various embodiments, isolating the one or more surface proteins on the LO comprises using flow cytometry.

In various embodiments, the biological sample is a liquid biopsy sample from the subject. In various embodiments, the biological sample is a plasma sample from the subject. In various embodiments, the biological sample is a serum sample from the subject.

### Quantification, Treatment

Various embodiments provide for a method of quantifying abundance of a cancer-derived protein signature in extracellular vesicles (EVs), comprising: detecting one or more surface proteins on large oncosomes (LOs), the one or more surface proteins selected from the group consisting of: (a) PIGS, CALR, CANX, LMAN2, RPN1, RPN2, VDAC1, STT3A and UGGT1, or (b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or (c) LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2; or (d) any combination of (a), (b), and/or (c); and quantifying the abundance of the one or more surface proteins.

Various embodiments provide for a method of quantifying abundance of a cancer-derived protein signature in extracellular vesicles (EVs), comprising: detecting one or more surface proteins on large oncosomes (LOs), the one or more surface proteins selected from the group consisting of: (a) PIGS, CALR, CANX, LMAN2, RPN1, RPN2, VDAC1, STT3A and UGGT1, or (b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or (c) LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1,ATP6AP1, ATP6VOA2, CRTAP, GLG1, GPR107, LMAB2, MMGT1, SSR1, TM9SF3, BRI3BP, CLPTM1, CLPTM1L, ERMP1, IRPR1, LEMD2, LMF2, MINPP1, PIGS, PNPLA6, TOR1AIP1, and TOR1AIP2, (e) TOMM40, SLC25A5, ATP5B, HSPA9, HSPD1, IMMT, and HADHA, or (f) ATP5B, HSPD1, RPN2, SLC25A5, TOMM40, DIP2B, and ERBB2IP; or (g) any combination of (a), (b), (c), (e), and/or (f); and quantifying the abundance of the one or more surface proteins.

Various embodiments provide for a method for treating cancer in a subject, comprising: obtaining the results of an analysis one or more proteins from large oncosomes (LO) isolated from a subject, wherein the one or more proteins are selected from the group consisting of: (a) PIGS, CALR, CANX, LMAN2, RPN1, RPN2, VDAC1, STT3A and UGGT1, or (b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or (c) LAMA5, CALR, YBX1, UBAI, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6V0A2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2; or (d) any combination of (a), (b), and/or (c); and managing the subject's cancer with an aggressive cancer treatment if one or more proteins are detected. In various embodiments, managing the subject's cancer with an aggressive cancer treatment comprises administering the aggressive cancer treatment.

Various embodiments provide for a method for treating cancer in a subject, comprising: obtaining the results of an analysis one or more proteins from large oncosomes (LO) isolated from a subject, wherein the one or more proteins are selected from the group consisting of: (a) PIGS, CALR, CANX, LMAN2, RPN1, RPN2, VDAC1, STT3A and UGGT1, or (b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or (c) LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2, or (e) TOMM40, SLC25A5, ATP5B, HSPA9, HSPD1, IMMT, and HADHA, or (f) ATP5B, HSPD1, RPN2, SLC25A5, TOMM40, DIP2B, and ERBB2IP; or (g) any combination of (a), (b), (c), (e), and/or (g); and managing the subject's cancer with an aggressive cancer treatment if one or more proteins are detected. In various embodiments, managing the subject's cancer with an aggressive cancer treatment comprises administering the aggressive cancer treatment.

Examples of aggressive cancer treatment include but are not limited to first and second line androgen deprivation therapy (ADT) and chemotherapy. An example of chemotherapy includes but is not limited to docetaxel.

In various embodiments, the biological sample was pelleted at about 2,800×g (2.8K) or at about 10,000×g (10K). In various embodiments, the biological sample was pelleted at about 2,800×g (2.8K) to 10,000×g (10K). In various embodiments, the biological sample was pelleted at about 2,500×g, about 5,000×g, about 7500×g, about 10,000×g, or about 12,500×g.

In various embodiments, the one or more proteins is selected from the group consisting of: any combination of (a), (b), and/or (c). In various embodiments, the one or more surface proteins is selected from the group consisting of: any combination of (e), and/or (f).

In various embodiments, the one or more proteins is selected from the group consisting of: PIGS, CALR, CANX, LMAN2, RPN1, RPN2, VDAC1, STT3A and UGGT1. In various embodiments, the one or more proteins is 2, 3, 4, 5, 6, 7, 8, or 9 of these proteins.

In various embodiments, the one or more proteins is selected from the group consisting of: ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3. In various embodiments, the one or more proteins is 2, 3, 4, 5, 6, 7, 8, 9, 10, 11, 12, 13, 14, 15, 16, 17, 18, 19, 20, 21, 22, 23,or 24 of these proteins.

In various embodiments, the one or more proteins is selected from the group consisting of: LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2. In various embodiments, the one or more proteins is 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36,37, 38, 39, 40, 41, 42, 43, 44 or 45 of these proteins.

In various embodiments, the one or more surface proteins is selected from the group consisting of: TOMM40, SLC25A5, ATP5B, HSPA9, HSPD1, IMMT, and HADHA. In various embodiments, the one or more surface proteins is 2, 3, 4, 5, 6, or 7 of these proteins.

In various embodiments, the one or more surface proteins is selected from the group consisting of: ATP5B, HSPD1, RPN2, SLC25A5, TOMM40, DIP2B, and ERBB2IP. In various embodiments, the one or more surface proteins is 2, 3, 4, 5, 6, or 7 of these proteins.

In various embodiments, the cancer is breast cancer. In various embodiments, the cancer is prostate cancer. In various embodiments, the caner is glioma.

Various embodiments of the present invention provide for a method for treating prostate cancer in a subject, comprising: detecting one or more surface proteins on large oncosomes (LO) isolated from a subject, wherein the one or more surface proteins are selected from the group consisting of LAMA5, LAMB2, RPN2, PIGS, and combinations thereof; managing the subject's prostate cancer with watchful waiting if LAMA5, LAMB2, or both are detected; or managing the subject's prostate cancer with an aggressive cancer treatment if RPN2, PIGS, or both are detected. In various embodiments, managing the subject's cancer with an aggressive cancer treatment comprises administering the aggressive cancer treatment. Examples of aggressive cancer treatment include but are not limited to first and second line androgen deprivation therapy (ADT) and chemotherapy. An example of chemotherapy includes but is not limited to docetaxel.

Various embodiments of the present invention provide a method for treating prostate cancer in a subject, comprising: obtaining the results of an analysis one or more surface proteins on large oncosomes (LO) isolated from a subject, wherein the one or more surface proteins are selected from the group consisting of LAMA5, LAMB2, RPN2, PIGS, and combinations thereof; and managing the subject's prostate cancer with watchful waiting if LAMA5, LAMB2, or both are detected; or managing the subject's prostate cancer with an aggressive cancer treatment if RPN2, PIGS, or both are detected. In various embodiments, managing the subject's cancer with an aggressive cancer treatment comprises administering the aggressive cancer treatment. Examples of aggressive cancer treatment include but are not limited to first and second line androgen deprivation therapy (ADT) and chemotherapy. An example of chemotherapy includes but is not limited to docetaxel.

Various embodiments of the present invention provide a method for treating prostate cancer in a subject, comprising: managing the subject's prostate cancer with watchful waiting if LAMA5, LAMB2, or both are detected on large oncosomes (LO) isolated from a subject, or managing the subject's prostate cancer with an aggressive cancer treatment if RPN2, PIGS, or both are detected on large oncosomes (LO) isolated from a subject. In various embodiments, managing the subject's cancer with an aggressive cancer treatment comprises administering the aggressive cancer treatment. Examples of aggressive cancer treatment include but are not limited to first and second line androgen deprivation therapy (ADT) and chemotherapy. An example of chemotherapy includes but is not limited to docetaxel.

As used herein, the term “label” refers to a composition capable of producing a detectable signal indicative of the presence of a target. Suitable labels include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means needed for the methods and devices described herein. For example, the peptides can be labeled with a detectable tag which can be detected using an antibody specific to the label.

Exemplary fluorescent labeling reagents include, but are not limited to, Hydroxycoumarin, Succinimidyl ester, Aminocoumarin, Methoxycoumarin, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5 or Cy7.

### Kits

The present invention is also directed to a kit to detect surface proteins and/or to identify and purify LOs. The kit is an assemblage of materials or components. Thus, in some embodiments the kit contains a composition including agents that specifically binds to one or more proteins on the surface or within the LOs and labels as described above.

The exact nature of the components configured in the inventive kit depends on its intended purpose. In one embodiment, the kit is configured particularly for the purpose of detection, identification, isolation, or purification for mammalian subjects. In another embodiment, the kit is configured particularly for the purpose of detection, identification, isolation, or purification for human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to detect, identify, isolate, or purify LOs having certain on the surface or within them. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

A variety of EV populations are present, in culture cell media and in body fluids, and differentiating one type from another can be important for at least three areas: 1) circulating biomarker discovery, 2) extracellular biology, and 3) Therapeutic doses and manufacturing.

We have previously reported that a population of cancer-specific EVs, large oncosomes (LO), can be isolated from conditioned cell culture media at 10,000×g. We noticed, however, that large EVs in the size range of LO can also be pelleted at a lower centrifugation speed of 2,800×g, and that this pellet is enriched in particularly large vesicles, 4-10 μm diameter, in comparison with the pellet obtained at 10,000×g. We optimized our EV isolation protocol to collect two large EV populations—pelleted at 2,800×g (2.8K) and 10,000×g (10K),—and a small EV population pelleted at 100,000×g (100K) and demonstrated that the proteins typically enriched in LO, HSPA5 and KT18, were present in the 2.8K and 10K pellets, and the proteins typically enriched in exosomes/small EVs, TSG101, CD81, and CD9 were present in the 100K pellet. (CAV1, which is expressed in EVs of different sizes and origin, was detected across all EV populations, and the intracellular marker CYC1 was detected only in the producing cells an absent in EVs).

We next set out to isolate and characterize the 2.8K, 10K, and 100K EV populations from three different cancer cell lines: prostate cancer (PC3), glioma (U87), and breast cancer (MDA-MB-231). Particle concentration in the EV preparations was quantified by Tunable Resistive Pulse Sensing (TRPS, qNano) and showed that, in each cell line, large EVs in the 2.8K and 10K pellets were 3-4 orders of magnitude less abundant than small EVs in the 100K pellets. The TRPS measurement of EV size distributions in each EV population showed very similar mode and mean diameters across the three cells lines. Transmission electron microscopy confirmed that the 2.8K and 10K EV preparations were enriched in large EVs and the 100K preparation was enriched in small EVs.

We performed untargeted label-free MS analysis of the three EV populations from PC3, U87, and MDA-MB-231 cells. The proteomic analysis revealed that, in each cell line, about one half of the detected proteins were common to all three EV populations. However, each EV population was also characterized by a set of proteins unique to it and a set of proteins shared with one other EV population. The 2.8K EVs had the highest number of proteins identified in it across all three cell lines. The 2.8K EVs from different cell lines also clustered together, based on their EV type, and not based on their cell line of origin.

To further characterize and compare the mRNA content of the 2.8K, 10K, and 100K EVs, we performed a comprehensive whole transcriptome analysis of the PC3 EVs and cells. In agreement with the proteomic data, transcriptomic analysis demonstrated that most of the detected transcripts were common to all three EV populations and to their producing cells, and each EV population, as well as producing cells, had a set of transcripts unique to it and a set of proteins shared with one another.

Unsupervised clustering analysis of the transcripts differentially enriched in the three EV populations and cells revealed that the most distinct RNA expression profiles were identified in the 2,8K and 100K EVs, and the 10K EVs appeared transitional between the other two. Interestingly, the RNA expression pattern in PC3 cells was more similar to the 2.8K EVs compared to the other two EV population.

The length of the RNA transcripts belonging to different biotypes was nearly identical in the PC3 cells and in all three EV populations; however, the frequency (abundance) of those transcripts was significantly lower in EVs compared to the cells.

The correlation between the proteins and protein-coding mRNA transcripts was higher in 2.8K vs 10K, and higher in 10K vs 100K, with the 2.8K and 10K EVs exhibiting the mRNA profiles more similar to each other than to the 100K EVs. Additionally, the protein cargo correlations between the 2.8K and 10K (0.64) and between the 10K and 100K (0.84) were lower than the mRNA correlations between these fractions (0.94 and 0.91, respectively), potentially indicating that the identity of the EV populations is defined by their protein cargo more than by their mRNA cargo. We also noticed that the mRNA transcripts in 2.8K and 10K are more similar to each other than the mRNA transcripts in 10K and 100K, while the proteins in 10K and 100K are more similar to each other than the proteins in 2.8K and 10K, indicating that 2.8K and 10K are more similar to each other at the mRNA level, whereas 10K and 100K are more similar at the protein level.

Gene ratio is the ratio of differentially expressed (input) genes out of all input genes that are represented in the term. On the proteome level, the pathway analysis demonstrated that proteins enriched in the 2.8K EVs are involved in biological processes related to protein metabolism and synthesis, as well as response to viral infection and extracellular matrix organization. Proteins enriched in 100K EVs are involved in cell adhesion, receptor-mediated signaling, and transmembrane ion transport.

On the transcriptome level, the pathway analysis demonstrated that the RNA transcripts enriched in the 2.8K EVs are involved in the biological processes related to RNA processing, mitochondrial organization and respiration, as well as response to viral infection-similar to the proteomic dataset. Similar to the transcripts enriched in the 2.8K EVs, the transcripts enriched in 100K EVs are also involved in the biological processes related to the RNA processing; however, the biological processes related to methylation, histone and protein modification were unique to it.

The 2.8K EVs from different cell lines cluster together, independently on the cell line of origin the most distinct RNA expression profiles were identified in the 2,8K vs 100K EVs, the 2,8K were more representative of the cell of origin. The 2,8K fraction also contained a slightly higher number of transcripts with >80% coverage the identity of the EV populations is defined by their protein cargo more than by their mRNA cargo.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. (canceled)

2. A method for isolating large oncosomes (LOs), comprising:
contacting a biological sample obtained from a subject to an agent that specifically binds to one or more surface proteins on the LOs, wherein the one or more surface proteins is selected from the group consisting of:
(a) PIGS, CALR, CANX, LMAN2, RPN1, RPN2, VDAC1, STT3A and UGGT1, or
(b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or
(c) LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2, or
(e) TOMM40, SLC25A5, ATP5B, HSPA9, HSPD1, IMMT, and HADHA, or
(f) ATP5B, HSPD1, RPN2, SLC25A5, TOMM40, DIP2B, and ERBB2IP; or
(g) any combination of (a), (b), (c), (e), and/or (f); and
isolating LOs having the one or more surface proteins.

3. The method of claim 2, further comprising identifying the subject as having cancer when one or more surface proteins are detected on the isolated LOs.

4. The method of claim 2, further comprising identifying the subject as having prostate cancer, glioma or breast cancer when one or more surface proteins are detected on the isolated LOs.

5. The method of claim 2, further comprising identifying the subject as having advance or aggressive cancer when one or more surface proteins are detected on the isolated LOs.

6. The method of claim 2, further comprising identifying the subject as having advance or aggressive prostate cancer, advance or aggressive glioma or advance or aggressive breast cancer when one or more surface proteins are detected on the isolated LOs.

7. The method of claim 5, further comprising managing the subject's cancer with an aggressive cancer treatment.

8. The method of claim 2, wherein the one or more surface proteins comprise LAMA5, LAMB2, or both.

9. The method of claim 8, further comprising identifying the subject as having stable prostate cancer.

10. The method of claim 9, further comprising managing the subject's prostate cancer with watchful waiting.

11. The method of claim 2, wherein the one or more surface proteins comprise RPN2, PIGS, or both.

12. The method of claim 11, further comprising identifying the subject as having a high likelihood of having lethal prostate cancer.

13. The method of claim 12, further comprising managing the subject's prostate cancer with an aggressive cancer treatment.

14. The method of any claim 2,
wherein the agent that specifically binds to one or more surface proteins on the LO is an antibody or binding fragment thereof that specifically binds to the one or more surface proteins on the LO, optionally, the antibody or binding fragment thereof is conjugated to a magnetic bead, or
wherein the agent that specifically binds to one or more surface proteins on the LO is affixed to a substrate, or
wherein the agent that specifically binds to one or more surface proteins on the LO is conjugated to a label.

15. (canceled)

16. (canceled)

17. The method of claim 14, wherein the label is a fluorescent label.

18. The method of claim 2,
wherein contacting the biological sample obtained from a subject to an agent that specifically binds to one or more surface proteins on the LO comprises using a cell sorter, or
wherein contacting the biological sample obtained from a subject to an agent that specifically binds to one or more surface proteins on the LO comprises using flow cytometry.

19. (canceled)

20. The method of claim 2, wherein the biological sample is a liquid biopsy sample from the subject, or wherein the biological sample is a plasma sample from the subject.

21. (canceled)

22-42. (canceled)

43. (canceled)

44. A method of quantifying abundance of a cancer-derived protein signature in extracellular vesicles (EVs), comprising:
detecting one or more surface proteins on large oncosomes (LOs), the one or more surface proteins selected from the group consisting of:
(a) PIGS, CALR, CANX, LMAN2, RPN1, RPN2, VDAC1, STT3A and UGGT1, or
(b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or
(c) LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2; or
(e) TOMM40, SLC25A5, ATP5B, HSPA9, HSPD1, IMMT, and HADHA, or
(f) ATP5B, HSPD1, RPN2, SLC25A5, TOMM40, DIP2B, and ERBB2IP; or
(g) any combination of (a), (b), (c), (e), and/or (f); and
quantifying the abundance of the one or more surface proteins.

45. (canceled)

46. A method for treating cancer in a subject, comprising:
obtaining the results of an analysis one or more surface proteins from large oncosomes (LO) isolated from a subject, wherein the one or more surface proteins are selected from the group consisting of
(a) PIGS, CALR, CANX, LMAN2, RPN1, RPN2, VDAC1, STT3A and UGGT1, or
(b) ACVR1, CLDN12, PTPRM, ROBO1, SLC23A2, ADAM10, ADAM17, ANO6, CD276, CD55, DAG1, DCBLD2, EPHA2, ITGA3, ITGA5, ITGB1, MFGE8, PLXNA1, PLXNB2, PTPRK, SLC44A1, SLC4A7, SLC1A5, and SLC5A3, or
(c) LAMA5, CALR, YBX1, UBA1, HNRNPK, KRT1, CAPNS1, ACTN1, GPI, SEC22B, RTN4, LRRC8A, FLNA, VDCA1, ACAA1, UGGT1, STT3A, ASPH, RPN2, RPN1, CANX, LMAN2, TMED9, SLC44A1, ATP6AP1, ATP6VOA2, CRTAP, GLG1, GPR107, LMAB2, MMGT1, SSR1, TM9SF3, BRI3BP, CLPTM1, CLPTM1L, ERMP1, ITPR1, LEMD2, LMF2, MINPP1, PIGS, PNPLA6, TOR1AIP1, and TOR1AIP2; or
(e) TOMM40, SLC25A5, ATP5B, HSPA9, HSPD1, IMMT, and HADHA, or
(f) ATP5B, HSPD1, RPN2, SLC25A5, TOMM40, DIP2B, and ERBB2IP; or
(g) any combination of (a), (b), (c), (e), and/or (f); and
managing the subject's cancer with an aggressive cancer treatment if one or more proteins are detected.

47. A method for treating prostate cancer in a subject, comprising:
detecting one or more surface proteins on large oncosomes (LO) isolated from a subject, wherein the one or more surface proteins are selected from the group consisting of LAMA5, LAMB2, RPN2, PIGS, and combinations thereof, or obtaining the results of an analysis one or more surface proteins on large oncosomes (LO) isolated from a subject, wherein the one or more surface proteins are selected from the group consisting of LAMA5, LAMB2, RPN2, PIGS, and combinations thereof; and
managing the subject's prostate cancer with watchful waiting if LAMA5, LAMB2, or both are detected, or
managing the subject's prostate cancer with an aggressive cancer treatment if RPN2, PIGS, or both are detected.

48. (canceled)

49. A method for treating prostate cancer in a subject, comprising:
managing the subject's prostate cancer with watchful waiting if surface proteins LAMA5, LAMB2, or both are detected on large oncosomes (LO) isolated from a subject, or
managing the subject's prostate cancer with an aggressive cancer treatment if surface proteins RPN2, PIGS, or both are detected on large oncosomes (LO) isolated from a subject.
